(19) United States
(12) Patent Application Publication
(10) Pub. No.: US 2026/0098005 A1
(43) Pub. Date: Apr. 9, 2026

(54) NON-FLUORINATED RESORCINOL AND HYDROQUINONE ANALOGS AS CURING AGENTS FOR FLUOROELASTOMERS

(71) Applicant: THE CHEMOURS COMPANY FC, LLC, WILMINGTON, DE (US)

(72) Inventors: PETER A. MORKEN, Wilmington, DE (US); ALEXANDER BORISOVICH SHTAROV, WILMINGTON, DE (US); ANDRII MATSNEV, THE PLAINS, OH (US); SEUNG KYUNG KO, SEOUL (KR)

(73) Assignee: THE CHEMOURS COMPANY FC, LLC, WILMINGTON, DE (US)

(21) Appl. No.: 19/112,767

(22) PCT Filed: Sep. 19, 2023
(86) PCT No.: PCT/US2023/033097

§ 371 Date: Mar. 18, 2025

Related U.S. Application Data
(60) Provisional application No. 63/408,361, filed on Sep. 20, 2022.

(51) Int. Cl. C07C 39/24; C07C 39/10; C08J 3/22

(52) U.S. Cl. CPC C07C 39/245; C07C 39/10; C08J 3/226; C08J 2327/12

(57) ABSTRACT

Compositions include a curing agent of Formula 1:

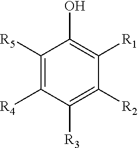

R₁ and R₅ are independently H, Cl, Br, C₁₋₁₈ alkyl or alkoxy which may contain chlorine or bromine substitutions, or X. R₂-R₄ are independently OH, H, Cl, Br, C₁₋₁₈ alkyl or alkoxy which may contain chlorine or bromine substitutions, or X. At least one of R₂, R₃, and R₄ is OH. Not more than 3 of R₁-R₅ are Cl or Br. X is Formula 2 or Formula 3:

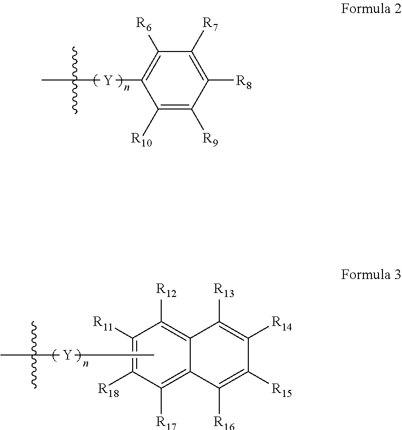

R₆-R₁₀ are independently H, Cl, Br, C₁₋₁₈ alkyl or alkoxy which may contain chlorine or bromine substitutions, acetyl or methylsulfonyl which may be alkyl- or aryl-substituted or may contain chlorine or bromine substitutions, nitro, nitrile, keto, aceto, or sulfone. R₁₁-R₁₈ are independently H, Cl, Br, C₁₋₁₈ alkyl or alkoxy which may contain chlorine or bromine substitutions, nitro, or nitrile, where one of R₁₁-R₁₈ is a single bond to —(Y)ₙ—. Y is —SO₂— or —O— and n is 0 or 1. At least one of R₁-R₅, is Cl, Br, C₁₋₁₈ alkyl or alkoxy which may contain chlorine or bromine substitutions, or X. When X is present, at least one of R₆-R₁₈ is Cl, Br, C₁₋₁₈ alkyl or alkoxy which may contain chlorine or bromine substitutions, nitro, nitrile, keto, aceto, or sulfone.

### REFERENCE TO RELATED APPLICATIONS

This application is a national filing under 35 U.S.C. 371 of International Application No. PCT/US2023/033097 filed Sep. 19, 2023, and claims priority to and the benefit of U.S. Provisional Application No. 63/408,361 filed Sep. 20, 2022, which is hereby incorporated by reference in its entirety.

### FIELD

This disclosure relates to the curing agents for fluoroelastomers and more particularly to non-fluorinated resorcinol and hydroquinone analogs useful as curing agents for fluoroelastomers.

### BACKGROUND

Fluoroelastomers have excellent heat resistance, oil resistance, and chemical resistance and have been widely used for sealing materials, containers, and hoses. Examples of fluoroelastomers include copolymers including monomer units of vinylidene fluoride (VF2) and monomer units of at least one other copolymerizable fluorine-containing monomer such as hexafluoropropylene (HFP), tetrafluoroethylene (TFE), chlorotrifluoroethylene (CTFE), vinyl fluoride (VF), or a fluorovinyl ether such as a perfluoro(alkyl vinyl ether) (PAVE). Specific examples of PAVE include perfluoro(methyl vinyl ether), perfluoro(ethyl vinyl ether), and perfluoro(propyl vinyl ether). Other fluoroelastomers include copolymers of TFE with a hydrocarbon olefin, such as ethylene or propylene. Perfluoroelastomers that are copolymers of TFE and PAVE are known.

In order to fully develop the physical properties of elastomers for use in molded elastomeric articles, the polymers must be cured, i.e., vulcanized or crosslinked. This is generally accomplished in connection with the molding process by mixing uncured polymer with a polyfunctional curing agent, heating and molding the mixture into the shape of the desired article, and then further heating the resultant molded mixture, thereby promoting a cross-linking reaction of the curing agent with the polymer to produce a cured fluoroelastomer article.

Certain grades of fluoroelastomers, for example certain copolymers of VF2/HFP or VF2/HFP/TFE that do not include a cure site monomer, are curable using a polyhydroxy compound as a curing agent. 2,2-bis(4-hydroxyphenyl)hexafluoropropane, often referred to as bisphenol AF (BPAF), is a widely used curing agent for polyhydroxy-curable grades of fluoroelastomers. As a curing agent for polyhydroxy-curable grades of fluoroelastomers, BPAF provides good processing of the fluoroelastomer during molding into articles and imparts good properties to cured fluoroelastomer articles.

With regard to processing during molding, BPAF does not cause excessive “scorch”, i.e., does not cure too quickly while the article is being molded, but provides desirable short curing times once curing of the article being molded begins. In fluoroelastomer articles after curing, BPAF imparts desirable low “compression set” properties to the fluoroelastomer. Compression set is a commonly used measurement of the ability of an elastomer to return to nearly its original thickness after being compressed at an elevated temperature.

There is currently a proposed restriction in the European Union that includes BPAF in a class of compounds with endocrine disrupting properties. Thus, the use of curing agents other than BPAF is desirable. In U.S. Pat. No. 6,610,790, a number of compounds are listed in addition to BPAF as curing agents for fluoroelastomers, including resorcinol, hydroquinone, and certain alkyl substituted resorcinols and hydroquinones. However, the other curing agents listed in In U.S. Pat. No. 6,610,790 do not provide the good processing and compression set properties that can be provided by BPAF.

### SUMMARY OF THE INVENTION

Non-fluorinated resorcinol and hydroquinone analogs disclosed herein provide a good balance of processability and compression set properties as alternative curing agents to BPAF in the curing of fluoroelastomers.

In one embodiment, a curable fluoroelastomer composition includes a polyhydroxy-curable fluoroelastomer, a curing agent of Formula 1, and an acid acceptor.

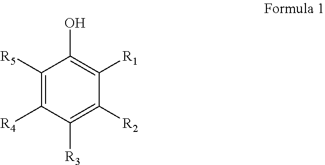

R₁ and R₅ are independently selected from the group consisting of H, Cl, Br, C₁₋₁₈ alkyl or alkoxy which may contain chlorine or bromine substitutions, and X, and R₂, R₃, and R₄ are independently selected from the group consisting of OH, H, Cl, Br, C₁₋₁₈ alkyl or alkoxy which may contain chlorine or bromine substitutions, and X, with the proviso that at least one of R₂, R₃, and R₄ is OH, and with the proviso that not more than 3 of R₁, R₂, R₃, R₄, and R₅ are Cl or Br.

X is selected from the group consisting of Formula 2 and Formula 3:

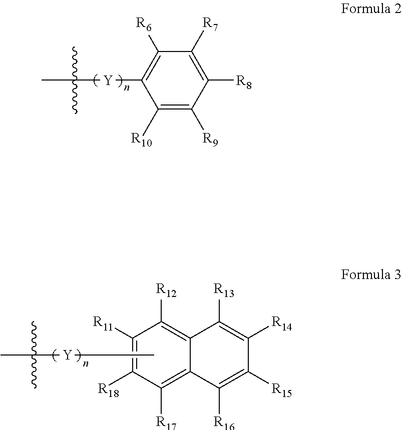

R₆, R₇, R₈, R₉, and Rio are independently selected from the group consisting of H, Cl, Br, C₁₋₁₈ alkyl or alkoxy which may contain chlorine or bromine substitutions, acetyl or methylsulfonyl which may be alkyl- or aryl-substituted or may contain chlorine or bromine substitutions, nitro, nitrile, keto, aceto, and sulfone; R₁₁, R₁₂, R₁₃, R₁₄, R₁₅, R₁₆, R₁₇, and R₁₈ are independently selected from the group consisting of H, Cl, Br, C₁₋₁₈ alkyl or alkoxy which may contain chlorine or bromine substitutions, nitro, and nitrile, with the proviso that one of R₁₁, R₁₂, R₁₃, R₁₄, R₁₅, R₁₆, R₁₇, and R₁₈ is a single bond to —(Y)ₙ—; Y is selected from the group consisting of —SO₂— and —O—; and n is 0 or 1; with the proviso that at least one of R₁, R₂, R₃, R₄, and R₅, is Cl, Br, C₁₋₁₈ alkyl or alkoxy which may contain chlorine or bromine substitutions, or X, and with the proviso that, when X is present, at least one of R₆, R₇, R₈, R₉, R₁₀, R₁₁, R₁₂, R₁₃, R₁₄, R₁₅, R₁₆, R₁₇, and R₁₈ is Cl, Br, C₁₋₁₈ alkyl or alkoxy which may contain chlorine or bromine substitutions, nitro, nitrile, keto, aceto, or sulfone.

In one embodiment of the composition, at least one of R₁ and R₅ is H.

In another embodiment of the composition, at least one of R₁, R₂, R₃, R₄, and R₅ is H when it is an adjacent substituent to OH.

In another embodiment of the composition, no more that one of R₁, R₂, R₃, R₄, and R₅ is X.

In another embodiment of the composition, no more than 2 of R₁, R₂, R₃, R₄, and R₅ are Cl or br.

In another embodiment of the composition, only one of R₂, R₃, and R₄ is OH.

In another embodiment of the composition, either R₂ or R₄ is OH.

In another embodiment of the composition, R₁ and R₅ are selected from the group consisting of H, Cl, Br, C₁₋₁₈ alkyl or alkoxy which may contain chlorine or bromine substitutions, and X; and R₂, R₃, and R₄ are independently selected from the group consisting of OH, H, Cl, Br, C₁₋₁₈ alkyl or alkoxy which may contain chlorine or bromine substitutions, and X.

In another embodiment of the composition, one of R₁, R₂, R₃, R₄, and R₅ is X, and X is Formula 2.

In another embodiment of the composition, R₆, R₇, R₈, R₉, and Rio are independently selected from the group consisting of H, Cl, Br, C₁₋₆ alkyl or alkoxy which may contain chlorine or bromine substitutions and at least one of R₆, R₇, R₈, R₉, and R₁₀ is Cl, Br, or C₁₋₆ alkyl or alkoxy which may contain chlorine or bromine substitutions.

In another embodiment of the composition, R₆, R₇, R₈, R₉, and Rio are independently selected from the group consisting of H, Cl, Br, and tertiary butyl and at least one of R₆, R₇, R₈, R₉, and Rio is Cl, Br, or tertiary butyl.

In another embodiment of the composition, n is 0.

In another embodiment of the composition, —Y— is —O—.

In another embodiment of the composition, the curing agent is selected from the group consisting of:

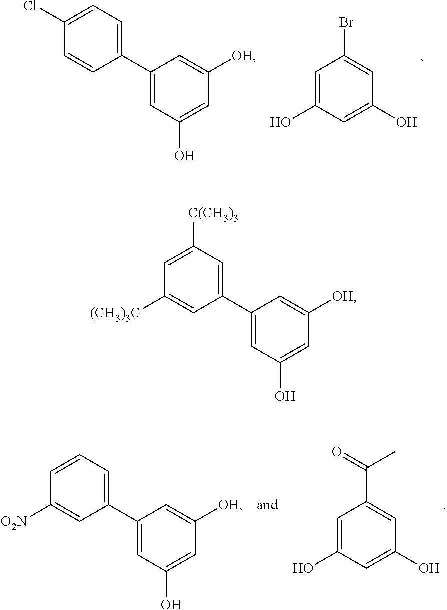 [SMILES: Oc1cc(O)cc(-c2ccc(Cl)cc2)c1 ; Oc1cc(O)cc(Br)c1 ; CC(C)(C)c1cc(-c2cc(O)cc(O)c2)cc(C(C)(C)C)c1 ; O=[N+]([O-])c1cccc(-c2cc(O)cc(O)c2)c1 ; "and" ; CC(=O)c1cc(O)cc(O)c1]

In another embodiment of the composition, the composition contains about 0.1 to about 10 parts by weight of said curing agent per 100 parts by weight of fluoroelastomer.

In another embodiment of the composition, the polyhydroxy-curable fluoroelastomer is a dipolymer of hexafluoropropylene and vinylidene fluoride.

In another embodiment of the composition, the acid acceptor is selected from the group consisting of powdered magnesium oxide, calcium hydroxide, and a combination thereof.

In another embodiment of the composition, the curable fluoroelastomer composition is free of 2,2 bis(4 hydroxyphenyl)hexafluoropropane.

In another embodiment, a fluoroelastomer masterbatch includes a polyhydroxy-curable fluoropolymer and curing agent of Formula 1. The curing agent is present at a concentration of about 1 wt % to about 50 wt %.

In one embodiment of the fluoroelastomer masterbatch, the concentration of said curing agent is about 20 wt % to about 40 wt %.

In another embodiment, a curing agent and curing accelerator mixture includes a curing agent of Formula 1 and a curing accelerator selected from the group consisting of a quaternary phosphonium salt, a quaternary ammonium salt, and a tertiary sulfonium salt.

In one embodiment of the curing agent and curing accelerator mixture, the curing accelerator is a tertiary sulfonium salt.

In another embodiment of the curing agent and curing accelerator mixture, the curing accelerator is a quaternary ammonium salt.

In another embodiment of the curing agent and curing accelerator mixture, the quaternary ammonium salt is tetrabutylammonium hydrogen sulfate.

In another embodiment of the curing agent and curing accelerator mixture, the curing accelerator is a quaternary phosphonium salt.

In another embodiment of the curing agent and curing accelerator mixture, the quaternary phosphonium salt is benzyl triphenyl phosphonium chloride.

In yet another embodiment, a salt for use as a fluoroelastomer curing agent and curing accelerator includes a quaternary phosphonium salt or quaternary ammonium salt derived from a compound of Formula 1.

In one embodiment of the salt, the curing accelerator is a quaternary ammonium salt.

In another embodiment of the salt, the quaternary ammonium salt is tetrabutylammonium hydrogen sulfate.

In another embodiment of the salt, the curing accelerator is a quaternary phosphonium salt.

In another embodiment of the salt, the salt is a benzyl triphenyl phosphonium salt.

In another embodiment, a method of curing a polyhydroxy-curable fluoroelastomer includes forming a curable fluoroelastomer composition including a polyhydroxy-curable fluoroelastomer, a curing agent of Formula 1, and an acid acceptor and heating the curable fluoroelastomer composition to cure the polyhydroxy-curable fluoroelastomer.

In one embodiment of the method, the curable fluoroelastomer composition is free of 2,2 bis(4 hydroxyphenyl)hexafluoropropane.

In another embodiment, an article is cured by the method.

In one embodiment of the article, the article is free of or substantially free of 2,2 bis(4 hydroxyphenyl)hexafluoropropane.

In another embodiment, a compound is of Formula 1A.

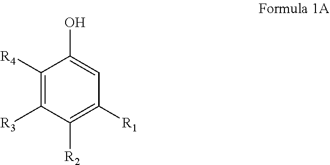

One of R₁ and R₂ is H and the other is OH. One of R₃ and R₄ is H and the other is Formula 2A:

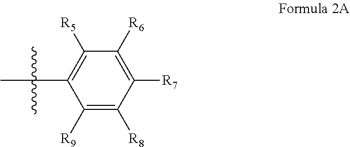

R₅, R₆, R₇, R₈, and R₉ are independently selected from the group consisting of H, Cl, Br, OCH₃, C(CH₃)₃, CH₃, nitro, nitrile, keto, aceto, and sulfone, with the proviso that when R₁ is OH and R₃ is Formula 2A: exactly one of R₅, R₆, R₇, R₈, and R₉ is selected from the group consisting of C(CH₃)₃, nitrile, aceto, and sulfone, and the remainder are H; exactly one of R₅, R₆, R₈, and R₉ is selected from the group consisting of nitro and keto, and the remainder of R₅, R₆, R₇, R₈, and R₉ are H; exactly one of R₅ and R₉ is CH₃ and the remainder of R₅, R₆, R₇, R₈, and R₉ are H; exactly one of R₆ and R₈ is Br and the remainder of R₅, R₆, R₇, R₈, and R₉ are H; or at least two of R₅, R₆, R₇, R₈, and R₉ are independently selected from the group consisting of Cl, OCH₃, C(CH₃)₃, nitro, nitrile, keto, aceto, and sulfone; with the proviso that when R₁ is OH and R₄ is Formula 2A: exactly one of R₅, R₆, R₇, R₈, and R₉ is selected from the group consisting of C(CH₃)₃, nitrile, keto, aceto, and sulfone, and the remainder are H; exactly one of R₅, R₆, R₈, and R₉ is selected from the group consisting of Cl, Br, OCH₃, and CH₃, and the remainder of R₅, R₆, R₇, R₈, and R₉ are H; exactly one of R₅, R₇, and R₅ is nitro, and the remainder of R₅, R₆, R₇, R₈, and R₉ are H; or at least two of R₅, R₆, R₇, R₈, and R₉ are independently selected from the group consisting of Cl, Br, OCH₃, C(CH₃)₃, CH₃, keto, aceto, and sulfone; and with the proviso that when R₂ is OH: exactly one of R₅, R₆, R₇, R₈, and R₉ is selected from the group consisting of keto and sulfone, and the remainder are H; exactly one of R₅, R₆, R₈, and R₉ is selected from the group consisting of C(CH₃)₃ and aceto, and the remainder of R₅, R₆, R₇, R₈, and R₉ are H; or at least two of R₅, R₆, R₇, R₈, and R₉ are independently selected from the group consisting of Cl, C(CH₃)₃, nitrile, keto, aceto, and sulfone.

In one embodiment of the compound, R₁ is OH and R₂ is H.

In another embodiment of the compound, R₁ is H and R₂ is OH.

Other features and advantages of the present invention will be apparent from the following more detailed description of the preferred embodiments, taken in conjunction with the accompanying drawings which illustrate, by way of example, the principles of the invention.

### DETAILED DESCRIPTION

Provided are exemplary non-fluorinated resorcinol and hydroquinone analogs that provide a good balance of processability and compression set properties as curing agents to replace BPAF in the curing of fluoroelastomers.

In exemplary embodiments, the curing agent provides cure properties and cured fluoroelastomer properties similar to BPAF as a curing agent. Such cure properties may be measured by a moving die rheometer (MDR) and may include, but are not limited to, the minimum S′ torque (M_{L}), the maximum S′ torque achieved during a specified time period (MH), the (scorch) time to increase one unit of S′ torque from M_{L} (ts1), the (scorch) time to increase two units of S′ torque from M_{L} (ts2), the (cure) time to an increase of 50% of S′ torque from M_{L} to M_{H} (t₅₀), and/or the (cure) time to an increase of 90% of S′ torque from M_{L} to M_{H} (t₉₀). Such cured fluoroelastomer properties may include, but are not limited to, compression set resistance, tensile strength (TS), the elongation at break (EB), and the elastic modulus at 100% (M100), and/or fluid aged properties.

In exemplary embodiments, the curing agent is of Formula 1:

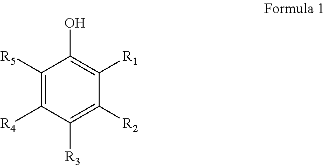

where R₁ and R₅ are independently selected from the group consisting of H, Cl, Br, C₁₋₁₈ alkyl or alkoxy which may contain chlorine or bromine substitutions, and X, and R₂, R₃, and R₄ are independently selected from the group consisting of OH, H, Cl, Br, C₁₋₁₈ alkyl or alkoxy which may contain chlorine or bromine substitutions, and X, with the proviso that at least one of R₂, R₃, and R₄ is OH, and with the proviso that not more than 3 of R₁, R₂, R₃, R₄, and R₅ are Cl or Br;
where X is selected from the group consisting of Formula 2 and Formula 3:

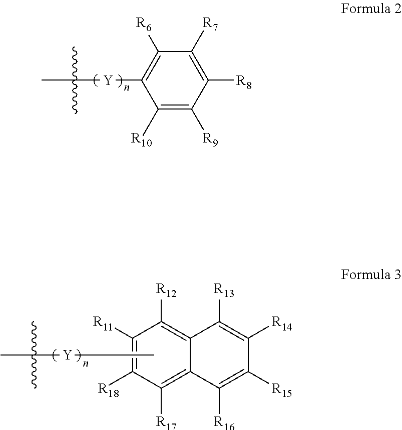

where R₆, R₇, R₈, R₉, and Rio are independently selected from the group consisting of H, Cl, Br, C₁₋₁₈ alkyl or alkoxy which may contain chlorine or bromine substitutions, acetyl or methylsulfonyl which may be alkyl- or aryl-substituted or may contain chlorine or bromine substitutions, nitro, nitrile, keto, aceto, and sulfone; R₁₁, R₁₂, R₁₃, R₁₄, R₁₅, R₁₆, R₁₇, and R₁₈ are independently selected from the group consisting of H, Cl, Br, C₁₋₁₈ alkyl or alkoxy which may contain chlorine or bromine substitutions, nitro, nitrile, keto, aceto, and sulfone, with the proviso that one of R₁₁, R₁₂, R₁₃, R₁₄, R₁₅, R₁₆, R₁₇, and R₁₈ is a single bond to —(Y)ₙ—; Y is selected from the group consisting of —SO₂— and —O—; and n is 0 or 1; with the proviso that at least one of R₁, R₂, R₃, R₄, and R₅, is Cl, Br, C₁₋₁₈ alkyl or alkoxy which may contain chlorine or bromine substitutions, or X, and with the proviso that, when X is present, at least one of R₆, R₇, R₈, R₉, R₁₀, R₁₁, R₁₂, R₁₃, R₁₄, R₁₅, R₁₆, R₁₇, and R₁₈ is Cl, Br, C₁₋₁₈ alkyl or alkoxy which may contain chlorine or bromine substitutions, nitro, nitrile, keto, aceto, or sulfone.

In some embodiments, at least one of R₁ and R₅ is H.

In some embodiments, at least one of R₁, R₂, R₃, R₄, and R₅ is H when it is an adjacent substituent to OH.

In some embodiments, no more that one of R₁, R₂, R₃, R₄, and R₅ is X.

In some embodiments, no more than 2 of R₁, R₂, R₃, R₄, and R₅ are Cl or Br.

In some embodiments, only one of R₂, R₃, and R₄ is OH.

In some embodiments, either R₂ or R₄ is OH.

In some embodiments, one of R₁, R₂, R₃, R₄, and R₅, is X and X is Formula 2.

In some embodiments, R₆, R₇, R₈, R₉, and Rio are independently selected from the group consisting of H, Cl, Br, C₁₋₆ alkyl which may contain chlorine or bromine substitutions, and C₁₋₆ alkoxy which contain chlorine or bromine substitutions and at least one of R₆, R₇, R₈, R₉, and Rio is Cl, Br, C₁₋₆ alkyl which may contain chlorine or bromine substitutions, or C₁₋₆ alkoxy which may contain chlorine or bromine substitutions.

In some embodiments, R₆, R₇, R₈, R₉, and Rio are independently selected from the group consisting of H, Cl, Br, and tertiary butyl and at least one of R₆, R₇, R₈, R₉, and Rio is fluorine, or tertiary butyl.

In some embodiments, n is 0.

In some embodiments, —Y— is —O—.

In some embodiments, the curing agent is a non-fluorinated resorcinol analog. Exemplary non-fluorinated resorcinol analogs may include, but are not limited to, the following structures:

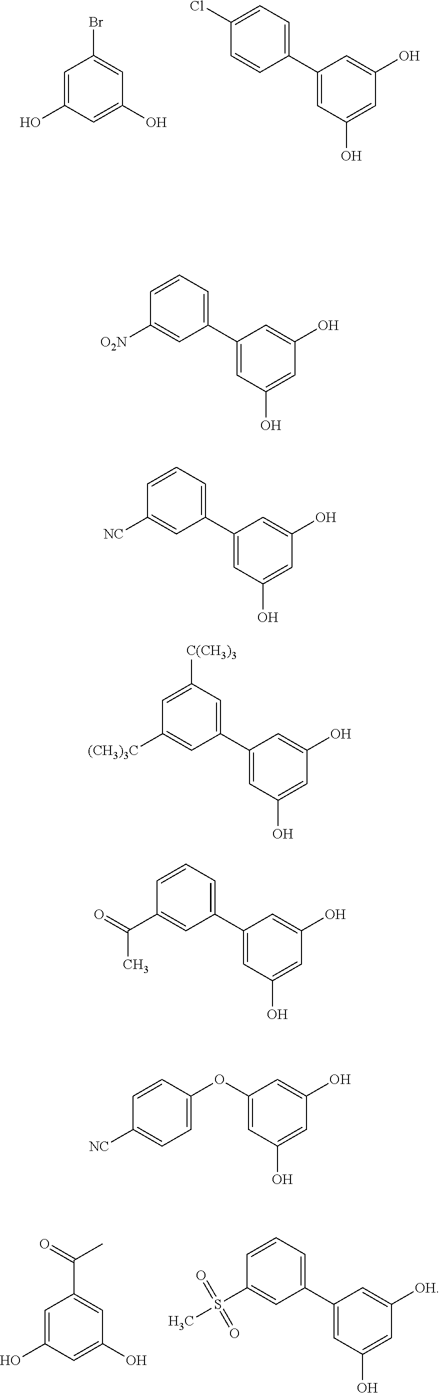

In some embodiments, the curing agent is a non-fluorinated hydroquinone analog. Exemplary non-fluorinated hydroquinone analogs include, but are not limited to, the following structures:

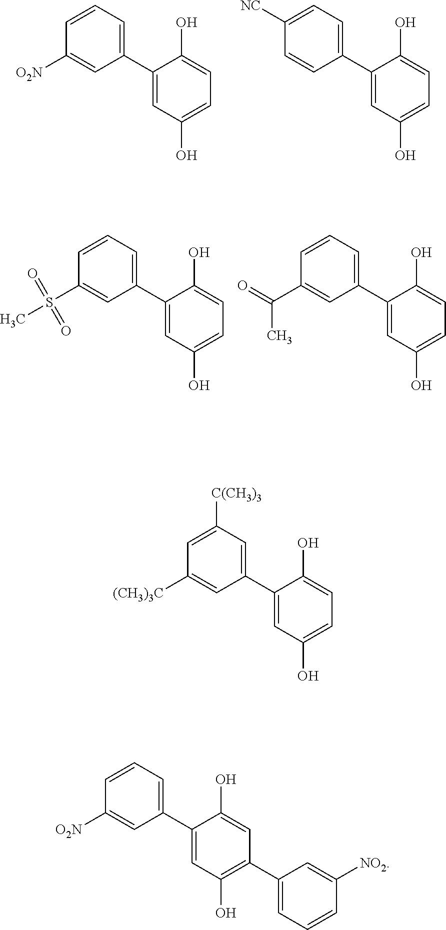

In some embodiments, the curing agent is selected from the following structures:

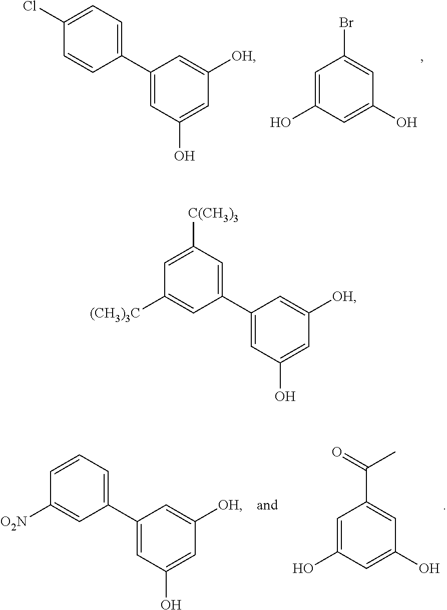 [SMILES: Oc1cc(O)cc(-c2ccc(Cl)cc2)c1 ; Oc1cc(O)cc(Br)c1 ; CC(C)(C)c1cc(-c2cc(O)cc(O)c2)cc(C(C)(C)C)c1 ; O=[N+]([O-])c1cccc(-c2cc(O)cc(O)c2)c1 ; "and" ; CC(=O)c1cc(O)cc(O)c1]

In some embodiments, the curing agent is part of a curable fluoroelastomer composition that further includes a polyhydroxy-curable fluoroelastomer and an acid acceptor.

In some embodiments, the curable fluoroelastomer composition includes about 0.1 to about 10 parts by weight of the curing agent per 100 parts by weight of fluoroelastomer, alternatively about 0.2 to about 5 parts by weight, alternatively about 0.5 to about 5 parts by weight, alternatively about 1 to about 2.4 parts by weight, or any value, range, or sub-range therebetween.

The fluoroelastomer may be any polyhydroxy-curable fluoroelastomer. As used herein, “polyhydroxy-curable” refers to fluoroelastomers that are known to crosslink with polyhydroxy curing agents such as BPAF. Such fluoroelastomers include, but are not limited to, those having a plurality of carbon-carbon double bonds along the main elastomer polymer chain and also fluoroelastomers that contain sites that may be readily dehydrofluorinated. The latter fluoroelastomers include, but are not limited to, those that contain adjacent copolymerized units of vinylidene fluoride (VF2) and hexafluoropropylene (HFP) as well as fluoroelastomers that contain adjacent copolymerized units of VF2 (or tetrafluoroethylene) and a fluorinated comonomer having an acidic hydrogen atom, such as, for example, 2-hydropentafluoropropylene; 1-hydropentafluoropropylene; trifluoroethylene; 2,3,3,3-tetrafluoropropene; or 3,3,3-trifluoropropene. Preferred fluoroelastomers include the copolymers of i) vinylidene fluoride with hexafluoropropylene and, optionally, tetrafluoroethylene (TFE); ii) vinylidene fluoride with a perfluoro(alkyl vinyl ether) such as perfluoro(methyl vinyl ether), 2-hydropentafluoropropylene and optionally, tetrafluoroethylene; iii) tetrafluoroethylene with propylene and 3,3,3-trifluoropropene; iv) tetrafluoroethylene, perfluoro(methyl vinyl ether) and hexafluoro-2-(pentafluorophenoxy)-1-(trifluorovinyloxy)propane, and v) ethylene with tetrafluoroethylene, perfluoro(methyl vinyl ether) and 3,3,3-trifluoropropylene. In some embodiments, the polyhydroxy-curable fluoroelastomer is a dipolymer of hexafluoropropylene and vinylidene fluoride. Polyhydroxy-curable fluoroelastomers may also include iodine or bromine-containing elastomers. For example, small amounts (0.01-1 wt %) of chlorine, bromine, or iodine can be introduced with telogens such as, for example, CH₂I₂ or I(CF₂)₄I, or monomers such as, for example, CH₂═CHCF₂CF₂X (X═Br, I) or chlorotrifluoroethylene. In some embodiments, the polyhydroxy-curable fluoroelastomer contains a bis-olefin, such as, for example, CH₂═CH(CF₂)ₙCH═CH₂ (where n=2-8) or CF₂=CFO(CF₂)ₙOCF═CF₂ (where n=2-8).

Appropriate acid acceptors may include, but are not limited to, powdered magnesium oxide, calcium hydroxide, zinc oxide, bismuth oxide, lead oxide, calcium oxide, hydrotalcite, barium carbonate, calcium carbonate, alkyl stearates, or a combination thereof. In some embodiments, the curable fluoroelastomer composition includes about 3 to about 15 parts by weight of the acid acceptor per 100 parts by weight of fluoroelastomer, alternatively about 5 to about 15 parts by weight, alternatively about 6 to about 12 parts by weight, alternatively about 8 to about 10 parts by weight, or any value, range, or sub-range therebetween. In some embodiments, a composition includes two or more acid acceptors.

In some embodiments the curable composition includes an organic base. Appropriate organic bases may include, but are not limited to, 1,8-diazobicyclo[5,4,0]undec-7-ene (DBU) or salts thereof, 1,5-diazabicyclo(4.3.0)-non-5-ene (DBN) or salts thereof, or a combination thereof.

In some embodiments, the curable composition includes one or more additives. Appropriate additives may include, but are not limited to, processing aids and/or colorants.

In some embodiments, a fluoroelastomer masterbatch includes the curing agent and a polyhydroxy-curable fluoropolymer.

In some embodiments, a curing agent and curing accelerator mixture includes the curing agent and a curing accelerator.

Appropriate curing accelerators may include, but are not limited to, tertiary sulfonium salts such as [(C₆H₅)₂S⁺(C₆H₁₃)][Cl]⁻, and [(C₆H₁₃)₂S(C₆H₅)]⁺[CH₃CO₂]⁻ and quaternary ammonium, phosphonium, arsonium, and stibonium salts of the formula R₅R₆R₇R₈Y⁺X⁻, where Y is phosphorous, nitrogen, arsenic, or antimony; R₅, R₆, R₇, and R₈ are individually C₁-C₂₀ alkyl, aryl, aralkyl, alkenyl, and the chlorine, fluorine, bromine, cyano, —OR, and —COOR substituted analogs thereof, with R being C₁-C₂₀ alkyl, aryl, aralkyl, alkenyl, and where X is halide, hydroxide, sulfate, sulfite, carbonate, pentachlorothiophenolate, tetrafluoroborate, hexafluorosilicate, hexafluorophosphate, dimethyl phosphate, and C₁-C₂₀ alkyl, aryl, aralkyl, and alkenyl carboxylates and dicarboxylates. Particularly preferred are benzyltriphenylphosphonium chloride, benzyltriphenylphosphonium bromide, tetrabutylammonium hydrogen sulfate, tetrabutylammonium hydroxide, tetrapropylammonium hydroxide, tetrabutylammonium bromide, tributylallylphosphonium chloride, tributyl-2-methoxypropylphosphonium chloride, 1,8-diazabicyclo[5.4.0]undec-7-ene, and benzyldiphenyl(dimethylamino)phosphonium chloride. Other appropriate curing accelerators include methyltrioctylammonium chloride, methyltributylammonium chloride, tetrapropylammonium chloride, benzyltrioctylphosphonium bromide, benzyltrioctylphosphonium chloride, methyltrioctylphosphonium acetate, tetraoctylphosphonium bromide, methyltriphenylarsonium tetrafluoroborate, tetraphenylstibonium bromide, 4-chlorobenzyltriphenyl phosphonium chloride, 8-benzyl-1,8-diazabicyclo(5.4.0)-7-undecenonium chloride, diphenylmethyltriphenylphosphonium chloride, allyltriphenyl-phosphonium chloride, tetrabutylphosphonium bromide, m-trifluoromethyl-benzyltrioctylphosphonium chloride, and other quaternary compounds disclosed in U.S. Pat. Nos. 5,591,804; 4,912,171; 4,882,390; 4,259,463; 4,250,278 and 3,876,654.

In some embodiments, the curing accelerator includes a quaternary phosphonium salt, a quaternary ammonium salt, or a tertiary sulfonium salt.

In some embodiments, the curing accelerator includes benzyl triphenyl phosphonium chloride or tetrabutylammonium hydrogen sulfate.

In some embodiments, the curing agent and the curing accelerator in the curing agent and curing accelerator mixture are at a weight ratio in the range of about 1:1 to about 12:1, alternatively about 1.5:1 to about 10:1, alternatively about 2:1 to about 8:1, or any value, range, or sub-range therebetween.

In some embodiments, a phenoxide derived from a curing agent of Formula 1 is in the form of a quaternary phosphonium salt or quaternary ammonium salt for use as a fluoroelastomer curing agent and curing accelerator.

In some embodiments, a process cures a polyhydroxy-curable fluoropolymer with the curing agent.

In some embodiments, the process includes forming a mixture of the polyhydroxy-curable fluoropolymer, the curing agent, at least one acid acceptor, and a curing accelerator.

In some embodiments, the mixture includes about 0.05 to about 1.5 parts by weight of the curing accelerator per 100 parts by weight of fluoroelastomer, alternatively about 0.1 to about 1 part by weight, alternatively about 0.2 to about 0.8 parts by weight, alternatively about 0.25 to about 0.65 parts by weight, or any value, range, or sub-range therebetween.

In some embodiments, the mixture further comprises a filler. The filler may one or more inorganic fillers, one or more polymeric fillers, or combinations thereof.

In some embodiments, the filler is a medium thermal carbon black. Other appropriate inorganic fillers may include, but are not limited to, silica, talc, titanium dioxide (TiO₂), barium sulfate (BaSO₄), calcium carbonate (CaCO₃), or a combination thereof. Appropriate polymeric fillers may include, but are not limited to, polytetrafluoroethylene (PTFE). In some embodiments, the mixture includes about 10 to about 40 parts by weight of the filler per 100 parts by weight of fluoroelastomer, alternatively about 20 to about 40 parts by weight, alternatively about 25 to about 35 parts by weight, alternatively about 30 parts by weight, or any value, range, or sub-range therebetween.

In some embodiments, the curing temperature is in the range of about 150° C. to about 200° C., alternatively about 160° C. to about 190° C., alternatively about 170° C. to about 180° C., or any value, range, or sub-range therebetween.

In some embodiments, the curing time is in the range of about 5 to about 60 minutes, alternatively about 5 to about 20 minutes, alternatively about 10 to about 30 minutes, alternatively about 20 to about 30 minutes, or any value, range, or sub-range therebetween.

In some embodiments, the curing agent provides cure properties that are similar to the cure properties of BPAF. Such properties may include, but are not limited to, M_{L}, M_{H}, ts1, ts2, t₅₀, and t₉₀. In some embodiments, the values are within 50%, alternatively within 40%, alternatively within 30%, alternatively within 20%, alternatively within 10%, alternatively within 5%, or any value, range, or sub-range therebetween, of the values for BPAF as a curing agent.

In some embodiments, the curing agent provides a cured fluoroelastomer having similar properties to a cured fluoroelastomer formed with BPAF as the curing agent. Such properties may include, but are not limited to, compression set resistance, tensile strength, elongation at break, and elastic modulus at 100%. In some embodiments, the values are within 50%, alternatively within 40%, alternatively within 30%, alternatively within 20%, alternatively within 10%, alternatively within 5%, or any value, range, or sub-range therebetween, of the values for BPAF as a curing agent.

In some embodiments, a method of curing a polyhydroxy-curable fluoroelastomer includes forming a curable fluoroelastomer composition including a polyhydroxy-curable fluoroelastomer, a curing agent of Formula 1, and an acid acceptor and heating the curable fluoroelastomer composition to cure the polyhydroxy-curable fluoroelastomer.

In some embodiments, the curable fluoroelastomer composition is free of or substantially free of 2,2 bis(4 hydroxyphenyl)hexafluoropropane.

In some embodiments, an article is cured by the method of curing.

In some embodiments, the article is free of or substantially free of 2,2 bis(4 hydroxyphenyl)hexafluoropropane.

In another embodiment, a compound is of Formula 1A:

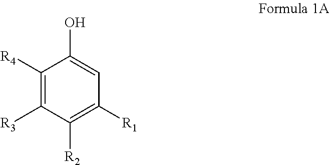

where one of R₁ and R₂ is H and the other is OH. One of R₃ and R₄ is H and the other is Formula 2A:

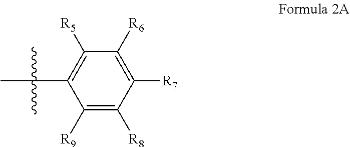

R₅, R₆, R₇, R₈, and R₉ are independently selected from the group consisting of H, Cl, Br, OCH₃, C(CH₃)₃, CH₃, nitro, nitrile, keto, aceto, and sulfone, with the proviso that when R₁ is OH and R₃ is Formula 2A: exactly one of R₅, R₆, R₇, R₈, and R₉ is selected from the group consisting of C(CH₃)₃, nitrile, aceto, and sulfone, and the remainder are H; exactly one of R₅, R₆, R₈, and R₉ is selected from the group consisting of nitro and keto, and the remainder of R₅, R₆, R₇, R₈, and R₉ are H; exactly one of R₅ and R₉ is CH₃ and the remainder of R₅, R₆, R₇, R₈, and R₉ are H; exactly one of R₆ and R₈ is Br and the remainder of R₅, R₆, R₇, R₈, and R₉ are H; or at least two of R₅, R₆, R₇, R₈, and R₉ are independently selected from the group consisting of Cl, OCH₃, C(CH₃)₃, nitro, nitrile, keto, aceto, and sulfone; with the proviso that when R₁ is OH and R₄ is Formula 2A: exactly one of R₅, R₆, R₇, R₈, and R₉ is selected from the group consisting of C(CH₃)₃, nitrile, keto, aceto, and sulfone, and the remainder are H; exactly one of R₅, R₆, R₈, and R₉ is selected from the group consisting of Cl, Br, OCH₃, and CH₃, and the remainder of R₅, R₆, R₇, R₈, and R₉ are H; exactly one of R₅, R₇, and R₅ is nitro, and the remainder of R₅, R₆, R₇, R₈, and R₉ are H; or at least two of R₅, R₆, R₇, R₈, and R₉ are independently selected from the group consisting of Cl, Br, OCH₃, C(CH₃)₃, CH₃, keto, aceto, and sulfone; and with the proviso that when R₂ is OH: exactly one of R₅, R₆, R₇, R₈, and R₉ is selected from the group consisting of keto and sulfone, and the remainder are H; exactly one of R₅, R₆, R₈, and R₉ is selected from the group consisting of C(CH₃)₃ and aceto, and the remainder of R₅, R₆, R₇, R₈, and R₉ are H; or at least two of R₅, R₆, R₇, R₈, and R₉ are independently selected from the group consisting of Cl, C(CH₃)₃, nitrile, keto, aceto, and sulfone.

Applications of the cured fluoropolymers described herein may include, but are not limited to, sealing materials, shaft seals, o-rings, containers, hoses, or wearable applications, such as, for example, wristwatch bands.

In some embodiments, the fluoroelastomers described are blended with one or more other fluoroelastomers or polymers to form a polymer blend. Appropriate blend polymers include, but are not limited to, nylon or other polyamides.

Although the curing agents have been described herein for curing curable fluoroelastomers, the curing agents may have other applications as well. In some embodiments, the curing agents are reacted with polyisocyanates to form polyurethanes.

In other embodiments, the curing agents are included in polyesters. In some such embodiments, the curing agents are condensed with aliphatic dicarboxylic acids or aromatic dicarboxylic acids, such as, for example, terephthalic acid, isophthalic acid, or mixtures thereof, or their esters to form aliphatic-aromatic polyesters or aromatic-aromatic polyesters, respectively. The resulting polymers may be amorphous, high-T_{g} materials or liquid crystalline aromatic polyesters. The introduction of the fluorinated aromatic side groups may result in good polymer processibility, good thermal stability, and/or good oxidative stability.

In other embodiments, the curing agents are included in polyimides, polyamides, polycarbonates, and/or epoxy resins.

### Test Methods

### Moving Die Rheometer (MDR) Measurements

Cure properties were measured on fluoroelastomer curing compositions of about 8 grams following ASTM D5289 on an MDR-2000 Rheometer (Alpha Technologies, Bellingham, WA). The curing temperature was 177° C., and the curing time was 24 minutes. The moving die frequency was 1.66 Hz, and the oscillation amplitude was 0.5°.

Reported cure properties include M_{L} in dN·m, M_{H} in dN·m, ts1 in minutes, ts2 in minutes, t₅₀ in minutes, and t₉o in minutes.

### Fluoroelastomer Property Measurements

Compression set resistances were determined on the fluoroelastomers with a compression device that compressed fluoroelastomer samples to 25% deflection following ASTM D395, Test Method B. Prior to the compression set testing, the fluoroelastomer was post-cured for 16 hours at 232° C. The compression set resistance is reported as a percentage change in thickness after a predetermined time at a predetermined temperature. Three values are reported herein: at 70 hours at 200° C. (CS1), at 168 hours at 200° C. (CS2), and at 70 hours at 250° C. (CS3).

Tensile properties were determined on the unaged fluoroelastomers at 23° C. by the ISO 37:2005 C or 1 2008 testing protocol. Measured tensile properties included the tensile strength in MPa, the elongation at break in %, and the elastic modulus at 100% in MPa.

### EXAMPLES

### Synthesis Examples

Four non-fluorinated resorcinol or hydroquinone analogs were prepared for evaluation as curing agents. The chemical structures of these Inventive Examples are shown in Table 1.

TABLE 1
Chemical Formulas of Inventive Examples

| Inventive | |
|---|---|
| Example | Formula |
| 1 | 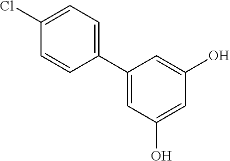 [SMILES: Oc1cc(O)cc(-c2ccc(Cl)cc2)c1] |
| 2 | 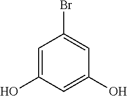 |
| 3 | 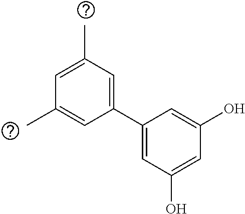 [SMILES: Cc1cc(C)cc(-c2cc(O)cc(O)c2)c1] |
| 4 | 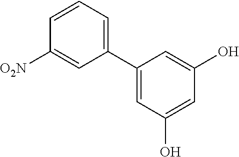 [SMILES: O=[N+]([O-])c1cccc(-c2cc(O)cc(O)c2)c1] |

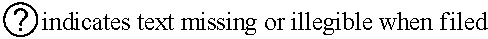

The non-fluorinated resorcinol analogs of Inventive Examples 1, 3, and 4 were prepared by a palladium catalyst-based synthesis approach often used for coupling of aryl-boronic acids with arylbromides as building blocks.

For synthesis of Inventive Example 1, 4-chloro-phenylboronic acid (13.5 g, 86.4 mmol), 1-bromo-3,5-dimethoxy-benzene (15 g, 69 mmol), potassium carbonate (17.2 g), Pd(PPh₃)₄ (0.4 g, 0.35 mmol), water (60 g), and toluene (180 g) were stirred and refluxed under nitrogen for 4 hours. The resulting toluene solution was separated, washed with water, and dried over MgSO₄. Toluene was removed by distillation, and the resulting (MeO)₂C₆H₃—C₆H₄-4-Cl was distilled under vacuum (145-150° C./1-1.3 Torr). The obtained (MeO)₂C₆H₃—C₆H₄-4-Cl intermediate (13.33 g, yield 78%, 53.6 mmol) was hydrolyzed with 48% hydrobromic acid (40.4 g) and acetic acid (45 g), at a temperature of 114° C. for 5 hours to achieve full conversion. A majority of the acids were removed by distillation in vacuum, and the distillation residue was neutralized with 9 g of 25% aqueous NaOH solution, extracted with ethyl acetate, washed with NaHCO₃ solution, and dried over MgSO₄. Ethyl acetate was removed by distillation to obtain the final pure 3,5-(HO)₂C₆H₃—C₆H₄-4-Cl (11.4 g, yield 96%, m.p.=141-144° C.) of Inventive Example 1 as a pale-yellow solid.

The material for Inventive Example 2 was acquired from Ambeed, Inc. (Arlington, IL, USA) and used without further purification.

For synthesis of Inventive Example 3, 15.2 g (110.1 mmol) of potassium carbonate and 0.64 g (0.55 mmol) of Pd(PPh₃)₄ were added under nitrogen to a mixture of 15 g (55.7 mmol) of 3,5-di-tert-butyl-bromobenzene and 10 g (55.0 mmol) of 3,5-dimethoxyphenylboronic acid in 150 mL of dioxane and 50 mL of deionized water. The reaction mixture was gently refluxed while stirring for 24 hours, and completion of the reaction was checked by TLC. Then, all solvent was evaporated, 300 mL of deionized water was added, and the crude material washed with water and dried. The crude material was redissolved in diethyl ether and filtered to remove catalyst, the solvent was evaporated, and the remaining material was recrystallized from hexane. 13.1 g (73.2%) of product was obtained. The obtained material (13 g, 40.0 mmol) was dissolved in 100 mL of DCM cooled in an ice bath, and 10 mL (105.4 mmol) of boron tribromide was added dropwise with intense stirring. The reaction mixture was allowed to slowly warm to room temperature and was left to stir overnight. The solvent was removed, and the reaction mixture was quenched with cold water. The crude product was extracted with dichloromethane. The solution was dried over magnesium sulfate and filtered, and the solvent evaporated. The residual material was recrystallized from hexane/ethyl acetate to give 10.3 g of Inventive Example 3 with a yield of 86.7%

For synthesis of the Inventive Example 4, 3-nitro-phenylboronic acid (24.5 g, 146.7 mmol), 1-bromo-3,5-dimethoxy-benzene (24.5 g, 113 mmol), potassium carbonate (28 g), Pd(PPh₃)₄ (0.65 g, 0.56 mmol), water (98 g), and toluene (220 g) were stirred and refluxed under nitrogen for 4 hours. The resulting toluene solution was separated, washed with water, and dried over MgSO₄. Toluene was removed by distillation and the resulting (MeO)₂C₆H₃—C₆H₄-3-NO₂ (20.8 g, yield 71.5%) was obtained as a distillation residue after the removal of more volatile components under vacuum (up to 190° C./0.6-0.9 Torr). The (MeO)₂C₆H₃—C₆H₄-4-Cl intermediate (19.5 g, 75.2 mmol) was hydrolyzed with 48% hydrobromic acid (97 g) and acetic acid (93 g), at a temperature of 114° C. for 5 hours and 116° C. for 3 hours. A majority of the acids were removed by distillation in vacuum, and the distillation residue was neutralized with 23 g of 20% aqueous NaOH solution, extracted with ethyl acetate, washed with NaHCO₃ solution, and dried over MgSO₄. Ethyl acetate was removed by distillation, and crude 3,5-(HO)₂C₆H₃—C₆H₄-3-NO₂ was purified by chromatography on silica gel and re-crystallized (4.0 g, yield 23%, m.p.=176-178° C.) of Inventive Example 4 was obtained as a yellow solid.

The melting points of the inventive examples were determined and are given in Table 2.

TABLE 2
Melting Points of Inventive Examples

| Inventive Example | Melting Point (° C.) |
|---|---|
| 1 | 141-144 |
| 2 | 87 |
| 3 | 176-177 |
| 4 | 176-178 |

### Comparative Fluoroelastomer Curing Examples

Since conditions for each set of curing runs was slightly different, 2,2-bis(4-hydroxyphenyl)hexafluoropropane (BPAF) (Comparative Examples A-D) was used as the curing agent for comparison for each set of curing runs for Inventive Examples of curing agents.

Comparative fluoroelastomer curing compositions included 100 parts by weight Viton™ A-500 (The Chemours Company FC LLC, Wilmington, DE) as the polyhydroxy-curable fluoroelastomer, 30 parts by weight medium thermal carbon black (MT Black) as a filler, 3 parts by weight powdered MgO (Elastomag® 170, Akrochem Corporation, Akron, OH) as an acid acceptor, 6 part by weight calcium hydroxide (Hallstar International, Chicago, IL) as an acid acceptor, 2 parts by weight BPAF, and 0.55 parts by weight benzyltriphenol phosphonium chlorite (BTPPC) as a curing accelerator.

### Inventive Fluoroelastomer Curing Examples

The prepared non-fluorinated resorcinol and hydroquinone analogs were evaluated as curing agents (Inventive Examples 1-4) in curing compositions.

Inventive fluoroelastomer curing compositions included 100 parts by weight Viton™ A-500 as the polyhydroxy-curable fluoroelastomer, 30 parts by weight MT Black as a filler, 3 parts by weight powdered MgO as an acid acceptor, 6 part by weight calcium hydroxide as an acid acceptor, 1.24 to 2.01 parts by weight curing agent, and 0.30 to 0.60 parts by weight BTPPC as a curing accelerator. The amounts of curing agent and BTPPC for each curing composition are shown in Table 3. The fluoroelastomer curing composition of Inventive Example 4 also included 1 part by weight of a processing aid.

TABLE 3
Fluoroelastomer Curing Compositions

| Inventive Example | Amount | BTPPC |
|---|---|---|
| 1 | 1.72 | 0.60 |
| 2 | 1.51 | 0.60 |
| 3 | 2.01 | 0.48 |
| 3 | 2.01 | 0.48 |
| 4 | 1.89 | 0.60 |

In some cases, several runs were made with the same curing agent, where the amount of curing agent and BTPPC were adjusted based on previous results to obtain cure properties and/or fluoroelastomer properties more similar to those with BPAF as the curing agent.

### Fluoroelastomer Curing Results

Cure properties for the Inventive Examples of Table 1 in the curing compositions of Table 3 and their respective Comparative Examples are shown in Table 4. Each Comparative Example is listed directly before the Inventive Example from the same set of MDR runs. When multiple runs were made with the same curing agent, only the run with the best combination of curing and fluoroelastomer properties was selected for inclusion in the Tables.

TABLE 4
Cure Properties

| Example | A | 1 | B | 2 | C | 3 | D | 4 |
|---|---|---|---|---|---|---|---|---|
| M_{L} (dN · m) | 0.94 | 1.05 | 0.91 | 1.32 | 0.82 | 0.71 | 0.83 | 0.83 |
| M_{H} (dN · m) | 23.78 | 23.98 | 23.84 | 25.46 | 22.58 | 22.92 | 23.13 | 23.45 |
| ts1 (min) | 1.21 | 1.38 | 1.13 | 0.77 | 1.03 | 1.11 | 1.10 | 1.38 |
| ts2 (min) | 1.25 | 1.62 | 1.27 | 0.89 | 1.13 | 1.28 | 1.24 | 1.65 |
| t₅₀ (min) | 1.56 | 2.16 | 1.58 | 1.41 | 1.39 | 1.71 | 1.57 | 2.34 |
| t₉₀ (min) | 2.15 | 2.90 | 2.20 | 2.78 | 1.85 | 2.36 | 2.52 | 3.84 |

Table 4 shows that the Inventive Examples provided cure properties that were similar to the cure properties of BPAF. Table 4 shows that for the Inventive Examples, M_{L} values were in the range of 0.71 to 1.32 dN·m, M_{H} values were in the range of 22.92 to 25.46 dN·m, ts1 values were in the range of 0.77 to 1.38 minutes, ts2 values were in the range of 0.89 to 1.65 minutes, t₅₀ values were in the range of 1.41 to 2.34 minutes, and t₅₀ values were in the range of 2.36 to 3.84 minutes.

### Fluoroelastomer Properties

Fluoroelastomer properties for the fluoroelastomers formed from the Inventive Examples of Table 1 in the curing compositions of Table 3 and the fluoroelastomers formed from their respective Comparative Examples are shown in Table 5. When multiple runs were made with the same curing agent, only the run with the best combination of curing and fluoroelastomer properties was selected for inclusion in the Tables.

TABLE 5
Fluoroelastomer Properties

| Example | A | 1 | B | 2 | C | 3 | D | 4 |
|---|---|---|---|---|---|---|---|---|
| TS [MPa] | 11.7 | 15.1 | 15.2 | 13.4 | 15.0 | 9.0 | 13.9 | 15.5 |
| EB [%] | 160 | 199 | 197 | 160 | 200 | 150 | 212 | 185 |
| M100 [MPa] | 6.3 | 6.9 | 6.3 | 7.3 | 6.1 | 5.5 | 5.1 | 6.9 |
| CS1 (%) | 17.4 | 23.3 | 18.6 | 25.6 | 19.8 | 23.3 | 17.4 | 24.4 |
| CS2 (%) | 27.9 | 36 | 30.2 | 37.2 | 30.2 | 35.6 | 26.7 | 36.5 |
| CS3 (%) | 58.1 | 67.4 | 62.8 | 81.4 | 66.3 | 70.1 | 62.8 | 68.2 |

Table 5 shows that the Inventive Examples provided a cured fluoroelastomer having similar properties to a cured fluoroelastomer formed with BPAF as the curing agent. Table 5 shows that for the Inventive Examples, TS values were in the range of 9.0 to 15.5 MPa, EB values were in the range of 150 to 199%, M100 values were in the range of 5.5 to 7.3 MPa, CS1 values were in the range of 23.3 to 25.6%, CS2 values were in the range of 35.6 to 37.2%, and CS3 values were in the range of 64.7 to 81.4%.

All above-mentioned references are hereby incorporated by reference herein.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A curable fluoroelastomer composition comprising:
a polyhydroxy-curable fluoroelastomer;
a curing agent of Formula 1: 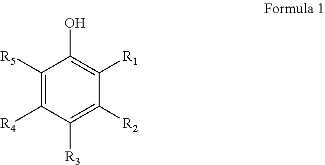
wherein R₁ and R₅ are independently selected from the group consisting of H, Cl, Br, C₁₋₁₈ alkyl which may contain chlorine or bromine substitutions, C₁₋₁₈ alkoxy which may contain chlorine or bromine substitutions, and X;
wherein R₂, R₃, and R₄ are independently selected from the group consisting of OH, H, Cl, Br, C₁₋₁₈ alkyl which may contain chlorine or bromine substitutions, C₁₋₁₈ alkoxy which may contain chlorine or bromine substitutions, and X,
with the proviso that at least one of R₂, R₃, and R₄ is OH;
with the proviso that not more than 3 of R₁, R₂, R₃, R₄, and R₅ are Cl or Br;
wherein X is selected from the group consisting of Formula 2 and Formula 3: 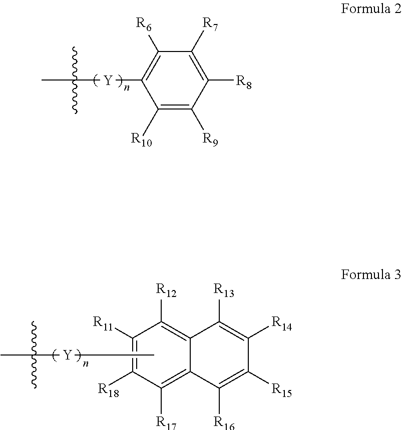
wherein R₆, R₇, R₈, R₉, and Rio are independently selected from the group consisting of H, Cl, Br, C₁₋₁₈ alkyl which may contain chlorine or bromine substitutions, C₁₋₁₈ alkoxy which may contain chlorine or bromine substitutions, acetyl or methylsulfonyl which may be alkyl- or aryl-substituted or may contain chlorine or bromine substitutions, nitro, nitrile, keto, aceto, and sulfone;
wherein R₁₁, R₁₂, R₁₃, R₁₄, R₁₅, R₁₆, R₁₇, and R₁₈ are independently selected from the group consisting of H, Cl, Br, C₁₋₁₈ alkyl which may contain chlorine or bromine substitutions, C₁₋₁₈ alkoxy which may contain chlorine or bromine substitutions, nitro, nitrile, keto, aceto, and sulfone with the proviso that one of R₁₁, R₁₂, R₁₃, R₁₄, R₁₅, R₁₆, R₁₇, and R₁₈ is a single bond to —(Y)ₙ—;
wherein Y is selected from the group consisting of —SO₂— and —O—; and
wherein n is 0 or 1;
with the proviso that at least one of R₁, R₂, R₃, R₄, and R₅, is Cl, Br, a chlorine-containing C₁₋₁₈ alkyl or alkoxy, a bromine-containing C₁₋₁₈ alkyl or alkoxy, or X;
with the proviso that, when X is present, at least one of R₆, R₇, R₈, R₉, R₁₀, R₁₁, R₁₂, R₁₃, R₁₄, R₁₅, R₁₆, R₁₇, and R₁₈ is Cl, Br, C₁₋₁₈ alkyl which may contain chlorine or bromine substitutions, C₁₋₁₈ alkoxy which may contain chlorine or bromine substitutions, nitro, nitrile, keto, aceto, or sulfone; and
an acid acceptor.

2. The curable fluoroelastomer composition of claim 1, wherein at least one of Ri and R₅ is H.

3. The curable fluoroelastomer composition of claim 1 or 2, wherein at least one of R₁, R₂, R₃, R₄, and R₅ is H when it is an adjacent substituent to OH.

4. The curable fluoroelastomer composition of any of the preceding claims, wherein no more that one of R₁, R₂, R₃, R₄, and R₅ is X.

5. The curable fluoroelastomer composition of any of the preceding claims, wherein no more than 2 of R₁, R₂, R₃, R₄, and R₅ are Cl or Br.

6. The curable fluoroelastomer composition of any of the preceding claims, wherein only one of R₂, R₃, and R₄ is OH.

7. The curable fluoroelastomer composition of claim 6, wherein either R₂ or R₄ is OH.

8. The curable fluoroelastomer composition of any of the preceding claims, wherein R₁ and R₅ are selected from the group consisting of H, Cl, Br, C₁₋₁₈ alkyl which may contain chlorine or bromine substitutions, C₁₋₁₈ alkoxy which may contain chlorine or bromine substitutions, and X; and wherein R₂, R₃, and R₄ are independently selected from the group consisting of OH, H, Cl, Br, C₁₋₁₈ alkyl which may contain chlorine or bromine substitutions, C₁₋₁₈ alkoxy which may contain chlorine or bromine substitutions, and X.

9. The curable fluoroelastomer composition of any of the preceding claims, wherein one of R₁, R₂, R₃, R₄, and R₅, is X and X is Formula 2.

10. The curable fluoroelastomer composition of claim 9 wherein R₆, R₇, R₈, R₉, and Rio are independently selected from the group consisting of H, Cl, Br, C₁₋₆ alkyl which may contain chlorine or bromine substitutions, and C₁₋₆ alkoxy which may contain chlorine or bromine substitutions, and at least one of R₆, R₇, R₈, R₉, and Rio is Cl, Br, C₁₋₆ alkyl which may contain chlorine or bromine substitutions, or C₁₋₆ alkoxy which may contain chlorine or bromine substitutions.

11. The curable fluoroelastomer composition of claim 9, wherein R₆, R₇, R₈, R₉, and Rio are independently selected from the group consisting of H, Cl, Br, and tertiary butyl, and at least one of R₆, R₇, R₈, R₉, and Rio is Cl, Br, or tertiary butyl.

12. The curable fluoroelastomer composition of any of the preceding claims, wherein n is 0.

13. The curable fluoroelastomer composition of any of the preceding claims, wherein —Y— is —O—.

14. The curable fluoroelastomer composition of claim 1, wherein the curing agent is selected from the group consisting of:

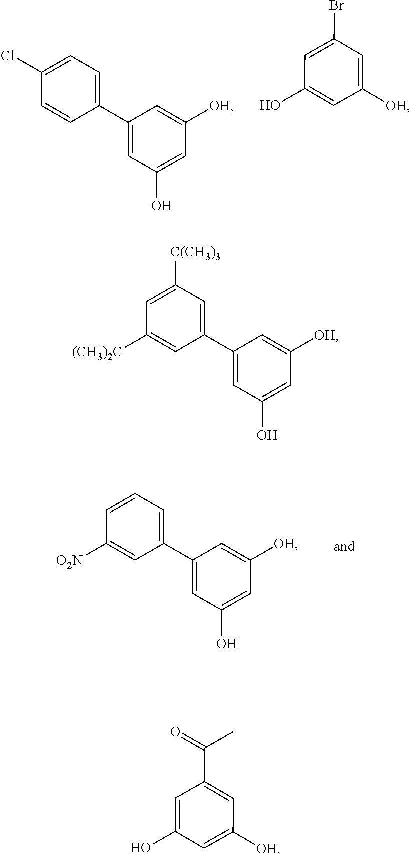

15. The curable fluoroelastomer composition of any of the preceding claims containing about 0.1 to about 10 parts by weight of said curing agent per 100 parts by weight of fluoroelastomer.

16. The curable fluoroelastomer composition of any of the preceding claims, wherein the polyhydroxy-curable fluoroelastomer is a dipolymer of hexafluoropropylene and vinylidene fluoride.

17. The curable fluoroelastomer composition of any of the preceding claims, wherein the acid acceptor is selected from the group consisting of powdered magnesium oxide, calcium hydroxide, and a combination thereof.

18. The curable fluoroelastomer composition of any of the preceding claims, wherein the curable fluoroelastomer composition is free of 2,2-bis(4-hydroxyphenyl)hexafluoropropane.

19. A fluoroelastomer masterbatch comprising a polyhydroxy-curable fluoropolymer and curing agent of Formula 1: 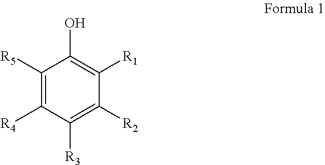
wherein R₁ and R₅ are independently selected from the group consisting of H, Cl, Br, C₁₋₁₈ alkyl which may contain chlorine or bromine substitutions, C₁₋₁₈ alkoxy which may contain chlorine or bromine substitutions, and X;
wherein R₂, R₃, and R₄ are independently selected from the group consisting of OH, H, Cl, Br, C₁₋₁₈ alkyl which may contain chlorine or bromine substitutions, C₁₋₁₈ alkoxy which may contain chlorine or bromine substitutions, and X,
with the proviso that at least one of R₂, R₃, and R₄ is OH;
with the proviso that not more than 3 of R₁, R₂, R₃, R₄, and R₅ are Cl or Br;
wherein X is selected from the group consisting of Formula 2 and Formula 3: 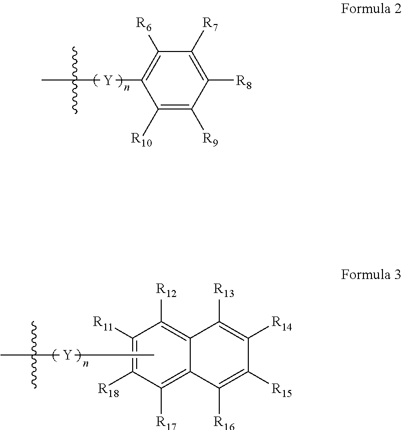
wherein R₆, R₇, R₈, R₉, and Rio are independently selected from the group consisting of H, Cl, Br, C₁₋₁₈ alkyl which may contain chlorine or bromine substitutions, C₁₋₁₈ alkoxy which may contain chlorine or bromine substitutions, acetyl or methylsulfonyl which may be alkyl- or aryl-substituted or may contain chlorine or bromine substitutions, nitro, nitrile, keto, aceto, and sulfone;
wherein R₁₁, R₁₂, R₁₃, R₁₄, R₁₅, R₁₆, R₁₇, and R₁₈ are independently selected from the group consisting of H, Cl, Br, C₁₋₁₈ alkyl which may contain chlorine or bromine substitutions, C₁₋₁₈ alkoxy which may contain chlorine or bromine substitutions, nitro, nitrile, keto, aceto, and sulfone with the proviso that one of R₁₁, R₁₂, R₁₃, R₁₄, R₁₅, R₁₆, R₁₇, and R₁₈ is a single bond to —(Y)ₙ—;
wherein Y is selected from the group consisting of —SO₂— and —O—; and
wherein n is 0 or 1;
with the proviso that at least one of R₁, R₂, R₃, R₄, and R₅, is Cl, Br, a chlorine-containing C₁₋₁₈ alkyl or alkoxy, a bromine-containing C₁₋₁₈ alkyl or alkoxy, or X;
with the proviso that, when X is present, at least one of R₆, R₇, R₈, R₉, R₁₀, R₁₁, R₁₂, R₁₃, R₁₄, R₁₅, R₁₆, R₁₇, and R₁₈ is Cl, Br, C₁₋₁₈ alkyl which may contain chlorine or bromine substitutions, C₁₋₁₈ alkoxy which may contain chlorine or bromine substitutions, nitro, nitrile, keto, aceto, or sulfone;
said curing agent being present at a concentration of about 1 wt % to about 50 wt %.

20. The fluoroelastomer masterbatch of claim 19, wherein the concentration of said curing agent is about 20 wt % to about 40 wt %.

21. A curing agent and curing accelerator mixture comprising a curing agent of Formula 1: 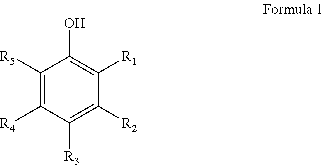
wherein R₁ and R₅ are independently selected from the group consisting of H, Cl, Br, C₁₋₁₈ alkyl which may contain chlorine or bromine substitutions, C₁₋₁₈ alkoxy which may contain chlorine or bromine substitutions, and X;
wherein R₂, R₃, and R₄ are independently selected from the group consisting of OH, H, Cl, Br, C₁₋₁₈ alkyl which may contain chlorine or bromine substitutions, C₁₋₁₈ alkoxy which may contain chlorine or bromine substitutions, and X,
with the proviso that at least one of R₂, R₃, and R₄ is OH;
with the proviso that not more than 3 of R₁, R₂, R₃, R₄, and R₅ are Cl or Br;
wherein X is selected from the group consisting of Formula 2 and Formula 3: 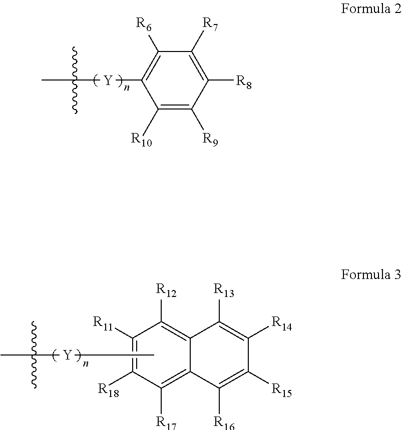
wherein R₆, R₇, R₈, R₉, and Rio are independently selected from the group consisting of H, Cl, Br, C₁₋₁₈ alkyl which may contain chlorine or bromine substitutions, C₁₋₁₈ alkoxy which may contain chlorine or bromine substitutions, acetyl or methylsulfonyl which may be alkyl- or aryl-substituted or may contain chlorine or bromine substitutions, nitro, nitrile, keto, aceto, and sulfone;
wherein R₁₁, R₁₂, R₁₃, R₁₄, R₁₅, R₁₆, R₁₇, and R₁₈ are independently selected from the group consisting of H, Cl, Br, C₁₋₁₈ alkyl which may contain chlorine or bromine substitutions, C₁₋₁₈ alkoxy which may contain chlorine or bromine substitutions, nitro, nitrile, keto, aceto, and sulfone with the proviso that one of R₁₁, R₁₂, R₁₃, R₁₄, R₁₅, R₁₆, R₁₇, and R₁₈ is a single bond to —(Y)ₙ—;
wherein Y is selected from the group consisting of —SO₂— and —O—; and
wherein n is 0 or 1;
with the proviso that at least one of R₁, R₂, R₃, R₄, and R₅, is Cl, Br, a chlorine-containing C₁₋₁₈ alkyl or alkoxy, a bromine-containing C₁₋₁₈ alkyl or alkoxy, or X;
with the proviso that, when X is present, at least one of R₆, R₇, R₈, R₉, R₁₀, R₁₁, R₁₂, R₁₃, R₁₄, R₁₅, R₁₆, R₁₇, and R₁₈ is Cl, Br, C₁₋₁₈ alkyl which may contain chlorine or bromine substitutions, C₁₋₁₈ alkoxy which may contain chlorine or bromine substitutions, nitro, nitrile, keto, aceto, or sulfone; and
a curing accelerator selected from the group consisting of a quaternary phosphonium salt, a quaternary ammonium salt, and a tertiary sulfonium salt.

22. The curing agent and curing accelerator mixture of claim 21, wherein the curing accelerator is a tertiary sulfonium salt.

23. The curing agent and curing accelerator mixture of claim 21, wherein the curing accelerator is a quaternary ammonium salt.

24. The curing agent and curing accelerator mixture of claim 23, wherein the quaternary ammonium salt is tetrabutylammonium hydrogen sulfate.

25. The curing agent and curing accelerator mixture of claim 21, wherein the curing accelerator is a quaternary phosphonium salt.

26. The curing agent and curing accelerator mixture of claim 25, wherein the quaternary phosphonium salt is benzyl triphenyl phosphonium chloride.

27. A salt for use as a fluoroelastomer curing agent and curing accelerator comprising a quaternary phosphonium salt or quaternary ammonium salt derived from a compound of Formula 1: 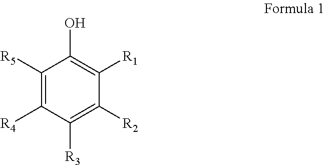
wherein R₁ and R₅ are independently selected from the group consisting of H, Cl, Br, C₁₋₁₈ alkyl which may contain chlorine or bromine substitutions, C₁₋₁₈ alkoxy which may contain chlorine or bromine substitutions, and X;
wherein R₂, R₃, and R₄ are independently selected from the group consisting of OH, H, Cl, Br, C₁₋₁₈ alkyl which may contain chlorine or bromine substitutions, C₁₋₁₈ alkoxy which may contain chlorine or bromine substitutions, and X,
with the proviso that at least one of R₂, R₃, and R₄ is OH;
with the proviso that not more than 3 of R₁, R₂, R₃, R₄, and R₅ are Cl or Br;
wherein X is selected from the group consisting of Formula 2 and Formula 3: 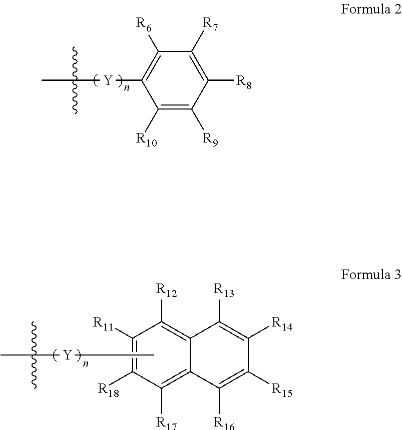
wherein R₆, R₇, R₈, R₉, and Rio are independently selected from the group consisting of H, Cl, Br, C₁₋₁₈ alkyl which may contain chlorine or bromine substitutions, C₁₋₁₈ alkoxy which may contain chlorine or bromine substitutions, acetyl or methylsulfonyl which may be alkyl- or aryl-substituted or may contain chlorine or bromine substitutions, nitro, nitrile, keto, aceto, and sulfone;
wherein R₁₁, R₁₂, R₁₃, R₁₄, R₁₅, R₁₆, R₁₇, and R₁₈ are independently selected from the group consisting of H, Cl, Br, C₁₋₁₈ alkyl which may contain chlorine or bromine substitutions, C₁₋₁₈ alkoxy which may contain chlorine or bromine substitutions, nitro, nitrile, keto, aceto, and sulfone with the proviso that one of R₁₁, R₁₂, R₁₃, R₁₄, R₁₅, R₁₆, R₁₇, and R₁₈ is a single bond to —(Y)ₙ—;
wherein Y is selected from the group consisting of —SO₂— and —O—; and
wherein n is 0 or 1;
with the proviso that at least one of R₁, R₂, R₃, R₄, and R₅, is Cl, Br, a chlorine-containing C₁₋₁₈ alkyl or alkoxy, a bromine-containing C₁₋₁₈ alkyl or alkoxy, or X;
with the proviso that, when X is present, at least one of R₆, R₇, R₈, R₉, R₁₀, R₁₁, R₁₂, R₁₃, R₁₄, R₁₅, R₁₆, R₁₇, and R₁₈ is Cl, Br, C₁₋₁₈ alkyl which may contain chlorine or bromine substitutions, C₁₋₁₈ alkoxy which may contain chlorine or bromine substitutions, nitro, nitrile, keto, aceto, or sulfone.

28. The salt of claim 27, wherein the curing accelerator is a quaternary ammonium salt.

29. The salt of claim 28, wherein the quaternary ammonium salt is tetrabutylammonium hydrogen sulfate.

30. The salt of claim 27, wherein the curing accelerator is a quaternary phosphonium salt.

31. The salt of claim 30, wherein the salt is a benzyl triphenyl phosphonium salt.

32. A method of curing a polyhydroxy-curable fluoroelastomer, the method comprising:
forming a curable fluoroelastomer composition comprising:
the polyhydroxy-curable fluoroelastomer;
a curing agent of Formula 1: 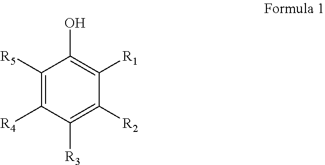
wherein R₁ and R₅ are independently selected from the group consisting of H, Cl, Br, C₁₋₁₈ alkyl which may contain chlorine or bromine substitutions, C₁₋₁₈ alkoxy which may contain chlorine or bromine substitutions, and X;
wherein R₂, R₃, and R₄ are independently selected from the group consisting of OH, H, Cl, Br, C₁₋₁₈ alkyl which may contain chlorine or bromine substitutions, C₁₋₁₈ alkoxy which may contain chlorine or bromine substitutions, and X,
with the proviso that at least one of R₂, R₃, and R₄ is OH;
with the proviso that not more than 3 of R₁, R₂, R₃, R₄, and R₅ are Cl or Br;
wherein X is selected from the group consisting of Formula 2 and Formula 3: 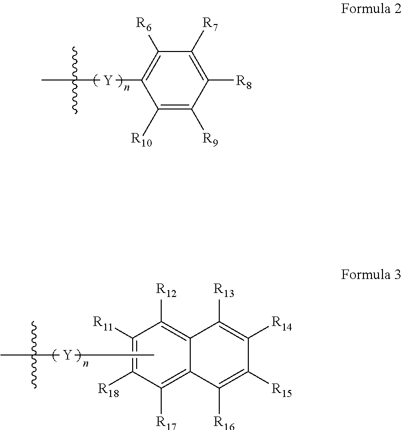
wherein R₆, R₇, R₈, R₉, and Rio are independently selected from the group consisting of H, Cl, Br, C₁₋₁₈ alkyl which may contain chlorine or bromine substitutions, C₁₋₁₈ alkoxy which may contain chlorine or bromine substitutions, acetyl or methylsulfonyl which may be alkyl- or aryl-substituted or may contain chlorine or bromine substitutions, nitro, nitrile, keto, aceto, and sulfone;
wherein R₁₁, R₁₂, R₁₃, R₁₄, R₁₅, R₁₆, R₁₇, and R₁₈ are independently selected from the group consisting of H, Cl, Br, C₁₋₁₈ alkyl which may contain chlorine or bromine substitutions, C₁₋₁₈ alkoxy which may contain chlorine or bromine substitutions, nitro, nitrile, keto, aceto, and sulfone with the proviso that one of R₁₁, R₁₂, R₁₃, R₁₄, R₁₅, R₁₆, R₁₇, and R₁₈ is a single bond to —(Y)ₙ—;
wherein Y is selected from the group consisting of —SO₂— and —O—; and
wherein n is 0 or 1;
with the proviso that at least one of R₁, R₂, R₃, R₄, and R₅, is Cl, Br, a chlorine-containing C₁₋₁₈ alkyl or alkoxy, a bromine-containing C₁₋₁₈ alkyl or alkoxy, or X;
with the proviso that, when X is present, at least one of R₆, R₇, R₈, R₉, R₁₀, R₁₁, R₁₂, R₁₃, R₁₄, R₁₅, R₁₆, R₁₇, and R₁₈ is Cl, Br, C₁₋₁₈ alkyl which may contain chlorine or bromine substitutions, C₁₋₁₈ alkoxy which may contain chlorine or bromine substitutions, nitro, nitrile, keto, aceto, or sulfone; and
an acid acceptor; and
heating the curable fluoroelastomer composition to cure the polyhydroxy-curable fluoroelastomer.

33. The method of claim 32, wherein the curable fluoroelastomer composition is free of 2,2-bis(4-hydroxyphenyl)hexafluoropropane.

34. An article cured by the method of claim 32.

35. The article of claim 34, wherein the article is free of or substantially free of 2,2-bis(4-hydroxyphenyl)hexafluoropropane.

36. A compound of Formula 1A: 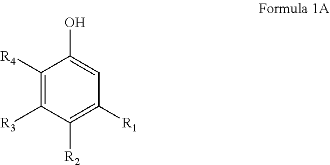
wherein one of R₁ and R₂ is H and the other is OH;
wherein one of R₃ and R₄ is H and the other is Formula 2A: 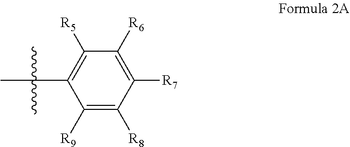
wherein R₅, R₆, R₇, R₈, and R₉ are independently selected from the group consisting of H, Cl, Br, OCH₃, C(CH₃)₃, CH₃, nitro, nitrile, keto, aceto, and sulfone;
with the proviso that when R₁ is OH and R₃ is Formula 2A:
exactly one of R₅, R₆, R₇, R₈, and R₉ is selected from the group consisting of C(CH₃)₃, nitrile, aceto, and sulfone, and the remainder are H;
exactly one of R₅, R₆, R₈, and R₉ is selected from the group consisting of nitro and keto, and the remainder of R₅, R₆, R₇, R₈, and R₉ are H;
exactly one of R₅ and R₉ is CH₃ and the remainder of R₅, R₆, R₇, R₈, and R₉ are H;
exactly one of R₆ and R₈ is Br and the remainder of R₅, R₆, R₇, R₈, and R₉ are H; or
at least two of R₅, R₆, R₇, R₈, and R₉ are independently selected from the group consisting of Cl, OCH₃, C(CH₃)₃, nitro, nitrile, keto, aceto, and sulfone;
with the proviso that when R₁ is OH and R₄ is Formula 2A:
exactly one of R₅, R₆, R₇, R₈, and R₉ is selected from the group consisting of C(CH₃)₃, nitrile, keto, aceto, and sulfone, and the remainder are H;
exactly one of R₅, R₆, R₈, and R₉ is selected from the group consisting of Cl, Br, OCH₃, and CH₃, and the remainder of R₅, R₆, R₇, R₈, and R₉ are H;
exactly one of R₅, R₇, and R₉ is nitro, and the remainder of R₅, R₆, R₇, R₈, and R₉ are H; or
at least two of R₅, R₆, R₇, R₈, and R₉ are independently selected from the group consisting of Cl, Br, OCH₃, C(CH₃)₃, CH₃, keto, aceto, and sulfone; and
with the proviso that when R₂ is OH:
exactly one of R₅, R₆, R₇, R₈, and R₉ is selected from the group consisting of keto and sulfone, and the remainder are H;
exactly one of R₅, R₆, R₈, and R₉ is selected from the group consisting of C(CH₃)₃ and aceto, and the remainder of R₅, R₆, R₇, R₈, and R₉ are H; or
at least two of R₅, R₆, R₇, R₈, and R₉ are independently selected from the group consisting of Cl, C(CH₃)₃, nitrile, keto, aceto, and sulfone.

37. The compound of claim 36, wherein R₁ is OH and R₂ is H.

38. The compound of claim 36, wherein R₁ is H and R₂ is OH.
